(19) United States
(12) Patent Application Publication
(10) Pub. No.: US 2023/0200683 A1
(43) Pub. Date: Jun. 29, 2023

(54) APPARATUS AND METHOD FOR SELF-TILT TRAINING

(71) Applicant: AJOU UNIVERSITY INDUSTRY-ACADEMIC COOPERATION FOUNDATION, Suwon-si, Gyeonggi-do (KR)

(72) Inventors: Jun Young CHOI, Yongin-si (KR); Tae Joon KIM, Seoul (KR)

(73) Assignee: AJOU UNIVERSITY INDUSTRY-ACADEMIC COOPERATION FOUNDATION, Suwon-si, Gyeonggi-do (KR)

(21) Appl. No.: 17/924,806

(22) PCT Filed: May 7, 2021
(86) PCT No.: PCT/KR2021/005712

§ 371 Date: Nov. 11, 2022

(30) Foreign Application Priority Data
May 11, 2020 (KR) 10-2020-0956187
Apr. 27, 2021 (KR) 10-2021-0054302

(51) Int. Cl. A61B 5/11; A61B 5/00; A61B 5/021; G16H 20/30; G16H 40/63

(52) U.S. Cl. CPC A61B 5/1113; A61B 5/4806; A61B 5/7275; A61B 5/021; G16H 20/30; G16H 40/63

(57) ABSTRACT

A self-tilt training apparatus according to an exemplary embodiment of the present disclosure includes a storage unit which stores a daily training setting value including a training tilt angle and a training time of a patient, a first sensor unit which includes a plurality of sensors in close contact with the patient and measures the tilt angle of the patient and a motion of the patient, a control unit which determines whether the patient stands up according to the set training tilt angle at the time of starting the training and whether the patient maintains the tilt angle during the training time on the basis of the daily training setting value and the measurement values of the first sensor unit, and an output unit which outputs a message, through any one or more manners of sight, touch, and hearing, according to a direction of the control unit.

### TECHNICAL FIELD

The present disclosure relates to an apparatus and a method for self-tilt training.

### BACKGROUND ART

The tilt training is utilized as a non-pharmacological treatment which treats recurrence of syncope by suppressing the hypersensitive response of the body's autonomic nervous system by giving the repeated stimulation from the outside in the neurocardiogenic syncope.

According to the tilt training method, once the patient is fasted, the patient is fixed on a head-up tilt table to be maintained for 45 minutes at 70-degrees of the head-up tilt or maintained until severe pre-syncope symptoms or syncope occurs. Depending on the patient's condition, the head-up tilt test is repeated one or two times a day and total average 8 to 10 times. Accordingly, the patient needs to be tested as an outpatient or inpatient during the tilt training.

FIG. 1 is an example of a normal tilting table.

Referring to FIG. 1, the tilting table includes a bed on which a patient lies and a fixing unit which ties up the patient. The bed is provided to control the angle.

The tilting table is expensive equipment, and some hospitals may not have the tilting table due to the large size of the equipment so that a patient who has a difficulty in standing may have to go to a distant hospital equipped with a tilting table.

In the case of the Republic of Korea, since September 2019, (A) patients with neurocardiogenic syncope who do not respond to existing drug treatment or have side effects due to long-term drug treatment and (B) patients with spinal cord injury of cervical and pleural effusion at or above the T6 level, patients with brainstem stroke, and patients with orthostatic hypotension due to autonomic nervous system failure in multiple system atrophy patients were defined as the benefit standard for the tilt training. Accordingly, patients with syncope and orthostatic hypotension who do not meet the current insurance benefit system criteria, and more patients in various disease groups, such as orthostatic intolerance, postural orthostatic tachycardia syndrome, or chronic fatigue syndrome, for whom tilt training helps relieve symptoms need to pay expensive bills.

Accordingly, attempts have been made to do tilt training by themselves at home.

In general, the self-tilt training needs to be performed one or two times a day by maintaining an upper body leaned against the wall with a distance of 15 cm or 20 cm from the wall for 30 minutes without moving. However, the training depends on the actor so that the tilt angle may vary depending on the actors, and when the fainting occurs during the self-training, there may be a risk of injury. There is also a disadvantage in that the compliance with self-training entirely depends on the report of the actor.

Accordingly, there are demands on an apparatus and a method which correctly check the training tilt angle of the actor, train according to a program set for individual conditions, and send a warning when fainting occurs during the training. Further, there are demands on an apparatus and a method which maintain high compliance with self-training.

### DISCLOSURE

### Technical Problem

An object of the present disclosure is to provide an apparatus and a method which allow a patient who needs a tilt training to perform the self-tilt training with a correct posture and send a warning when fainting occurs.

Further, according to the exemplary embodiment of the present disclosure, a painting possibility during the self-tilt training is predicted to safely perform the training.

Further, according to the exemplary embodiment of the present disclosure, the training is encouraged to maintain a high compliance with the self-training, and the training record is automatically reported to a manager (a doctor and a guardian).

Problems to be solved by the present disclosure are not limited to the above-mentioned problem(s), and other problem(s), which is (are) not mentioned above, can be clearly understood by those skilled in the art from the following descriptions.

### Technical Solution

A self-tilt training apparatus according to an exemplary embodiment of the present disclosure includes a storage unit which stores a daily training setting value including a training tilt angle and a training time of a patient; a first sensor unit which includes a plurality of sensors in close contact with the patient and measures the tilt angle of the patient and a motion of the patient; a control unit which determines whether the patient stands up according to a set training tilt angle at the time of starting the training and whether the patient maintains the tilt angle during the training time on the basis of the daily training setting value and the measurement values of the first sensor unit; and an output unit which outputs a message, through any one or more manners of sight, touch, and hearing, according to a direction of the control unit.

In one exemplary embodiment, the self-tilt training apparatus further includes a communication unit which transmits a message to a predetermined terminal according to a direction of the control unit, and compares the matching of a tilt angle measured when the training starts and a set training tilt angle and outputs a message including a comparison result to any one or more of the communication unit and the output unit.

In one exemplary embodiment, the control unit determines a fainting state of the patient during the training time on the basis of the measurement value of the first sensor unit and outputs a message including a determination result through any one or more of the communication unit and the output unit.

In one exemplary embodiment, the self-tilt training apparatus further includes a second unit which measures any one or more of a blood pressure, a pulse, an electrocardiogram, a brainwave, and a cerebral blood flow of the patient, and the control unit predicts a risk of fainting of the patient on the basis of the measurement value of the second sensor unit and outputs a message including a prediction result to any one or more of the communication unit and the output unit.

In one exemplary embodiment, the self-tilt training apparatus when it is determined to correspond to any one or more of cases when the blood pressure corresponds to a predetermined abnormal state, when the pulse corresponds to a predetermined abnormal state, when the electrocardiogram is not a sinus rhythm, when a brain wave changes from an alpha wave to a theta wave or a delta wave, and when a mean velocity of the cerebral blood flow is reduced to 30% or more, predicts that the patient has a risk of fainting.

In one exemplary embodiment, a case when the blood pressure corresponds to a predetermined abnormal state is any one of cases when a systolic blood pressure after the tilt training is reduced by 20 or more than the systolic blood pressure before the tilt training, when a systolic blood pressure after the tilt training is lower than 90, and when a diastolic blood pressure after the tilt training is reduced by 10 or more than a diastolic blood pressure before the tilt training and a case when the pulse corresponds to a predetermined abnormal state includes a case when the pulse within 10 minutes after tilt training is increased by 30 or more from the pulse before the tilt training without reducing the blood pressure, a case when the pulse after tilt training is increased to 120 or more, and a case when the pulse is reduced to 40 or less per minute after the tilt training.

In one exemplary embodiment, the self-tilt training apparatus further includes an input unit which receives information from the patient and the storage unit acquires and stores a daily training setting value from any one of the communication unit and the input unit.

In one exemplary embodiment, the control unit records a training time and a training tilt angle generated according to the tilt training of the patient at every date in the type of calendar to output the training time and the training tilt angle through any one or more of the communication unit and the output unit.

In one exemplary embodiment, when a training time included in the daily training setting value is not fulfilled, the control unit outputs a message for encouraging the training through any one or more of the communication unit and the output unit.

In one exemplary embodiment, the first sensor unit includes a gyro sensor and an acceleration sensor, and the second sensor unit includes a vibration sensor, an acoustic sensor, a pulse measurement sensor, an electrocardiogram sensor, a blood pressure sensor, a respiration measurement sensor, an electroencephalogram sensor, and a cerebral blood flow sensor.

In one exemplary embodiment, the control unit outputs any one or more of a predetermined music and a motivation comment which are stored in advance during the training time through the output unit.

A self-tilt training method by an apparatus carried by a patient according to an exemplary embodiment of the present disclosure includes acquiring and storing a daily training setting value including a training tilt angle and a training time of the patient; measuring a tilt angle of the patient and a motion of the patient by a first sensor unit including a plurality of sensors in close contact with the patient; and determining whether the patient stands up according to a set training tilt angle at the time of starting the training and whether the patient maintains the tilt angle during the training time on the basis of the daily training setting value and the measurement values of the first sensor unit.

In one exemplary embodiment, the matching of a tilt angle measured when the training starts and a set training tilt angle is measured and a message including a comparison result is output through any one or more of the communication unit and the output unit.

In one exemplary embodiment, in the determining, when it is determined that the patient has fainted on the basis of the measurement value measured by the first sensor unit, a warning message is output through any one or more manners of sight, touch, and hearing, or transmitted to a predetermined external terminal.

In one exemplary embodiment, in the measuring of a motion of the patient includes: measuring any one or more of a blood pressure, a pulse, and a heart rate by a second sensor unit including one or more sensors, and when it is determined that the patient has fainted on the basis of the measurement value measured by the first sensor unit and the second sensor unit, a warning message is output through any one or more manners of sight, touch, and hearing, or transmitted to a predetermined external terminal.

In one exemplary embodiment, in the acquiring and storing of a daily training setting value, the daily training setting value is input from the user by means of the input unit or received and acquired from the external terminal.

In one exemplary embodiment, when a training time included in the daily training setting value is not completed, a warning message which provides a message for encouraging the training is output through any one or more manners of sight, touch, and hearing, or transmitted to a predetermined external terminal.

### Advantageous Effects

According to the exemplary embodiment of the present disclosure, using an apparatus and a method for self-tilt training, the present disclosure provides an apparatus and a method for self-tilt training so as to allow a patient to safely perform the self-tilt training with a correct posture.

Further, according to the exemplary embodiment of the present disclosure, the high compliance with the self-training may be maintained.

According to the exemplary embodiment of the present disclosure, the self-tilt training record is automatically reported to a manager (a doctor and a guardian) to provide the convenience of the patient and provide a treatment effect with a low cost.

### DESCRIPTION OF DRAWINGS

FIG. 1 is an example of a normal tilting table.

FIG. 2 is a conceptual view for explaining a self-tilt training system of a patient according to an exemplary embodiment of the present disclosure.

FIG. 3 is a block diagram schematically illustrating a configuration of an apparatus for self-tilt training according to an exemplary embodiment of the present disclosure.

FIG. 4 is a flowchart for explaining a self-tilt training method by a device possessed by a patient according to an exemplary embodiment of the present disclosure.

FIG. 5 is a flowchart for explaining a method for determining a patient posture and condition by a device possessed by a patient according to an exemplary embodiment of the present disclosure.

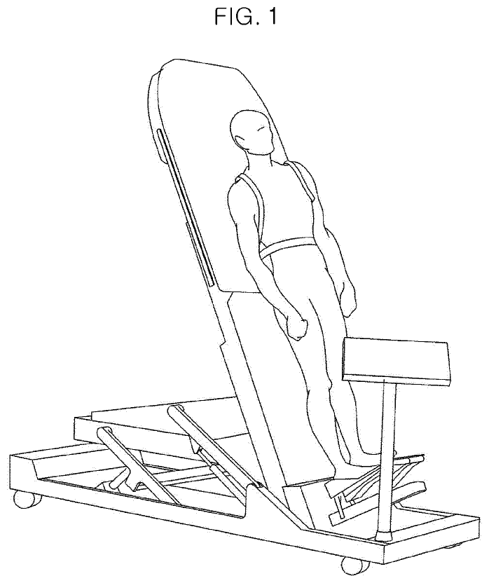

### BEST MODE

Those skilled in the art may make various modifications to the present disclosure and the present disclosure may have various embodiments thereof, and thus specific embodiments will be illustrated in the drawings and described in detail in detailed description. However, this does not limit the present disclosure within specific exemplary embodiments, and it should be understood that the present disclosure covers all the modifications, equivalents and replacements within the spirit and technical scope of the present disclosure. In the description of respective drawings, similar reference numerals designate similar elements.

Terms such as first, second, A, or B may be used to describe various components but the components are not limited by the above terms. The above terms are used only to distinguish one component from the other component. For example, without departing from the scope of the present disclosure, a first component may be referred to as a second component, and similarly, a second component may be referred to as a first component. A term of and/or includes combination of a plurality of related elements or any one of the plurality of related elements.

It should be understood that, when it is described that an element is “coupled” or “connected” to another element, the element may be directly coupled or directly connected to the other element or coupled or connected to the other element through a third element. In contrast, when it is described that an element is “directly coupled” or “directly connected” to another element, it should be understood that no element is not present therebetween.

Terms used in the present application are used only to describe a specific exemplary embodiment, but are not intended to limit the present disclosure. A singular form may include a plural form if there is no clearly opposite meaning in the context. In the present disclosure, it should be understood that terminology “include” or “have” indicates that a feature, a number, a step, an operation, a component, a part or the combination thereof described in the specification is present, but do not exclude a possibility of presence or addition of one or more other features, numbers, steps, operations, components, parts or combinations, in advance.

If it is not contrarily defined, all terms used herein including technological or scientific terms have the same meaning as those generally understood by a person with ordinary skill in the art. Terms defined in generally used dictionary shall be construed that they have meanings matching those in the context of a related art, and shall not be construed in ideal or excessively formal meanings unless they are clearly defined in the present application.

Hereinafter, an exemplary embodiment of the present disclosure will be described in detail with reference to the accompanying drawings.

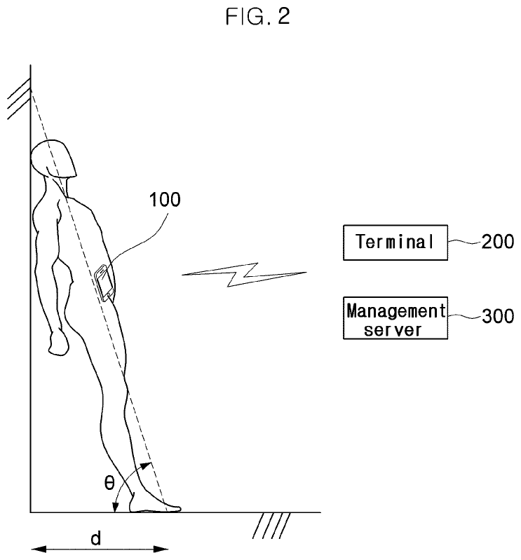

FIG. 2 is a conceptual view for explaining a self-tilt training system of a patient according to an exemplary embodiment of the present disclosure.

A patient mentioned in the exemplary embodiment of the present disclosure is a patient who requires tilt training.

The patient may perform the tilt training in a desired location using the self-tilt training apparatus 100.

The self-tilt training apparatus 100 is an apparatus which includes a plurality of sensors to measure a tilt angle and a motion, and outputs measured information.

The self-tilt training apparatus 100 is preferably lightweight to be easily carried for oneself during the training time.

The self-tilt training apparatus 100 may use a mobile phone, a smart phone, a PDA, a PMP, a WiBro-terminal, and a telematics device, but is not limited thereto, so that any device in which an application can be installed, data is transmitted/received by means of the application, and the received data can be displayed may be used. For example, it may be implemented in a wearable device, such as a smart band or a smart watch.

A patient may directly input a daily training setting value set for the patient's condition for the tilt training to the self-tilt training apparatus 100, or access a management server 300 in accordance with the instruction of a doctor to download and store the daily training setting value.

Next, the patient prepares for the tilt training in a place with a flat bottom and no dangerous object therearound to perform the training in a close contact with a body.

The patient may measure the tilt angle θ by means of the self-tilt training apparatus 100 with an upper body leaned against the wall with a predetermined distance d from the wall. The predetermined distance d may be approximately 15 cm or 20 cm, and the tilt angle is a tilt angle of the patient with respect to the ground.

The self-tilt training apparatus 100 notifies if the tilt angle of the patient does not match a predetermined daily training setting value, so that the user changes the posture correctly. If it is considered that the tilt angle is smaller than the daily training setting value, the self-tilt training apparatus 100 may guide the patient to move away from the wall or if it is considered that the tilt angle is larger than the daily training setting value, guide the patient to be closer to the wall. By doing this process, the patient may take an accurate posture according to the daily training setting value.

After taking the correct posture, the self-tilt training apparatus 100 monitors the motion and the tilt angle of the patient, and checks whether the posture of the patient is correct during the training time to output a message to take a correct posture if the posture of the patient is not correct.

The self-tilt training apparatus 100 may determine a fainting state based on a changed amount of the motion during the training time. If it is determined that the patient has fainted, the self-tilt training apparatus 100 may automatically transmit a message to a predetermined terminal 200 of a guardian, and so forth.

The self-tilt training apparatus 100 implementing the same may be specifically configured as follows.

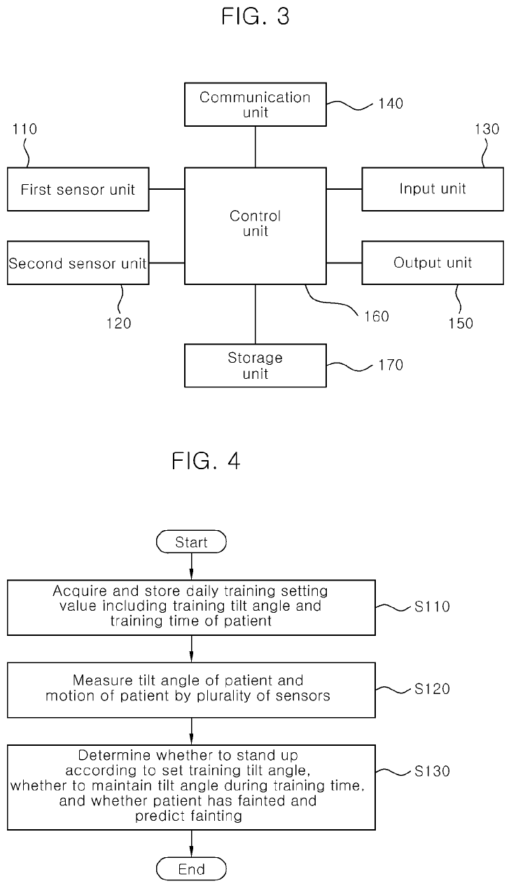

FIG. 3 is a block diagram schematically illustrating a configuration of a self-tilt training apparatus according to an exemplary embodiment of the present disclosure.

Referring to FIG. 3, the self-tilt training apparatus 100 according to an exemplary embodiment of the present disclosure may include a first sensor unit 110, a second sensor unit 120, an input unit 130, a communication unit 140, a storage unit 150, a control unit 160, and an output unit 170.

The first sensor unit 110 is configured by a plurality of sensors, and may include a sensor which measures a tilt of the patient and a sensor which measures a motion of the patient.

The first sensor unit 110 may include a gyro sensor and an acceleration sensor.

The second sensor unit 120 is configured by one or more sensors, and may further include a sensor which measures a biometric condition of the patient. The second sensor unit 120 may include any one or more of a vibration sensor, an acoustic sensor, a pulse sensor, an electrocardiogram sensor, a blood pressure sensor, a respiration sensor, an electroencephalogram sensor, and a cerebral blood flow sensor. The biometric condition of the patient may include any one or more of a blood pressure, a pulse, an electrocardiogram, a brain wave, and a cerebral blood flow.

The input unit 130 converts an input operation of the user into an input signal to transmit the input signal to the control unit 160. The input unit 130 may be implemented by a keyboard, a mouse, a touch sensor on a touch screen, a touch pad, a key pad, voice input, and other input processing devices which are available now, in the past or in the future. For example, the input unit 130 inputs a personal setting value including a daily training angle and a training time of the user to store the personal setting value in the storage unit 150.

The communication unit 140 transmits and receives information to and from an external terminal and a server. For example, the communication unit 140 receives the daily training setting value from the external terminal and the server to store the daily training setting value in the storage unit 150. Further, the communication unit 140 may transmit information generated during the training to a guardian's terminal and a medical staff's terminal which are set in advance according to the direction of the control unit 160. A communication technique used by the communication unit 140 may vary depending on a type of a communication network or other circumstances.

The storage unit 150 stores a program and data for providing a service for self-tilt training to the user. Specifically, the storage unit 150 may store the daily training setting value including a daily training angle and a training time for the tilt training. Further, the storage unit 150 stores information generated during the training, and may transmit data requested in accordance with the request of the control unit 160 to the control unit 160.

The control unit 160 controls an overall operation and each component of the self-tilt training apparatus 100. Specifically, the control unit 160 monitors whether a training tilt posture of the patient is correct based on measurement values measured by the first sensor unit 110 and the second sensor unit 120 based on the daily training setting value stored in the storage unit 150, and if the training tilt posture is not correct, transmits a warning message to cause the patient to take the training with the correct posture. The control unit monitors the condition of the patient based on the measurement values measured by the first sensor unit 110 and the second sensor unit 120, and when the fainting is predicted during the training, if it is determined that the posture of the training tilt angle of the patient is not correct, or it is determined that the patient has fainted, the control unit outputs a warning message through the output unit 170 or transmits the warning message to a predetermined terminal and a server by means of the communication unit 140. The predetermined terminal and server may be any one or more of terminals and servers of a guardian, an emergency treatment organization, and a doctor in charge.

Further, the control unit 160 may manage a training calendar in which the daily training setting value is recorded. The control unit 160 records information (training histories) generated during the training on the training calendar, and when a predetermined training time is not completed, outputs a message for encouraging the training through the output unit 170.

Further, when the training is finished, the control unit 160 automatically marks the training completion on the calendar to generate a sense of achievement.

Further, the control unit 160 outputs a previously stored music or a comment for encouraging or motivating the training during the normal training to improve the compliance of the training. The previously stored music may be a music including a frequency which calms the patient's mind.

The output unit 170 outputs a message, through any one or more manners of sight, touch, and hearing, according to a direction of the control unit. The output unit 170 may include any one or more of a display screen, a speaker, and a vibration sensor. The display screen may be implemented by a liquid crystal display device (LCD), a light emitting diode (LED), an organic light emitting diode (OLED), a projector, and other display devices which are available now, in the past or in the future.

Even though in the exemplary embodiment of the present disclosure, it is described that the second sensor unit is an integrated component of the self-tilt training apparatus 100, the second sensor unit 120 may be one component, and in this case, the second sensor unit 120 is implemented to transmit and receive information to and from the tilt training apparatus 100. For example, the self-tilt training apparatus 100 is a smart phone, and the second sensor unit 120 may be a wearable device such as a smart band. In some cases, the self-tilt training apparatus 100 may be implemented in a wearable form.

As described above, when the self-tilt training apparatus 100 transmits and receives data, depending on the viewpoint, it may be expressed that the communication unit 140 transmits and receives data in response to the control of the control unit 160, or the control unit 160 controls the communication unit 140 to transmit and receive data.

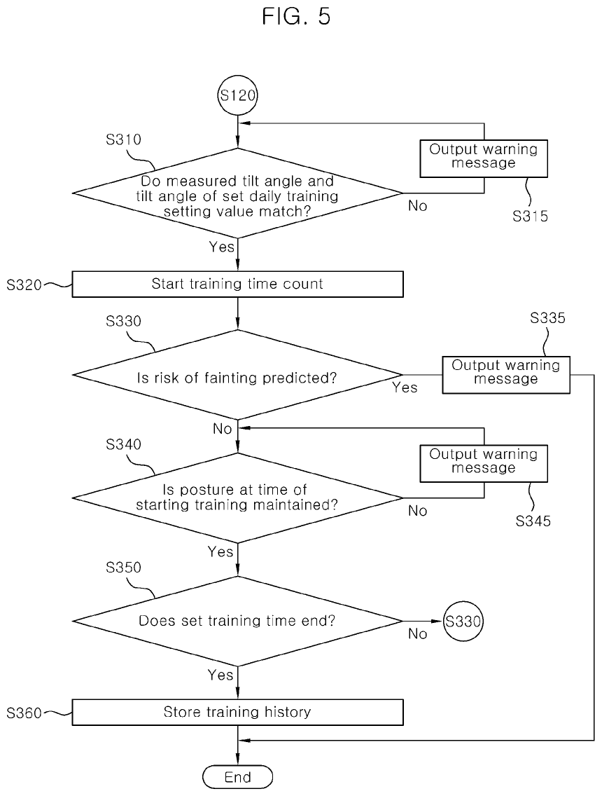

Specific operations of the components of the self-tilt training apparatus 100 will be described below with reference to FIGS. 4 and 5.

FIG. 4 is a flowchart for explaining a self-tilt training method by a device possessed by a patient according to an exemplary embodiment of the present disclosure, and FIG. 5 is a flowchart for explaining a method for determining a patient posture and condition by a device possessed by a patient according to an exemplary embodiment of the present disclosure.

In step S110, an apparatus acquires and stores a daily training setting value including a training tilt angle and a training time of a patient. A method for acquiring the daily training setting value has been described in detail with reference to FIG. 3 so that a redundant description will be omitted.

In step S120, the tilt angle of the patient and the motion of the patient are measured by the plurality of sensors.

At this time, any one or more of a blood pressure, a pulse, a respiration, and a heart rate may be further measured by the plurality of sensors.

In step S130, it is determined whether the patient stands up according to the set training tilt angle when the training starts, whether the patient maintains the tilt angle during the training time without moving, whether the patient has fainted, and whether there is a risk of fainting of the patient on the basis of the daily training setting value and measurement values measured by the plurality of sensors.

Referring to FIG. 5, in step S131, the apparatus compares the matching of the tilt angle measured by the plurality of sensors, for example, a gyro sensor and a tilt angle included in the set daily training setting value.

As a result of the comparison, if it is determined that the values do not match, the process goes to step S315 to output a warning message guiding to match the measured tilt angle and the set training tilt angle. Desirably, the apparatus may output a voice (auditory) to allow the patient to easily check the warning message, but is not limited thereto, and may transmit visual and touch information together.

As a result of the comparison, if it is determined that the values do not match, the process goes to step S320 to start the training timing count.

In one exemplary embodiment, any one or more of a previously stored music and a motivation comment are output through the output unit during the training time to improve the compliance of the patient.

In step S330, the risk of fainting of the patient is predicted through the measurement values by the plurality of sensors.

When it is determined to correspond to any one or more of cases when the blood pressure corresponds to a predetermined abnormal state, when a pulse corresponds to a predetermined abnormal state, when the electrocardiogram is not a sinus rhythm, when a brain wave changes from an alpha wave to a theta wave or a delta wave, or when a mean velocity of the cerebral blood flow is reduced to 30% or more, the patient may be predicted to have a risk of fainting.

Here, the abnormal state of the blood pressure is any one of cases when a systolic blood pressure after the tilt training is reduced by 20 or more from the systolic blood pressure before the tilt training, when a systolic blood pressure after the tilt training is lower than 90, or when a diastolic blood pressure after the tilt training is reduced by 10 or more from a diastolic blood pressure before the tilt training. Further, in the abnormal state of the pulse, the predetermined abnormal state is a case when the pulse within 10 minutes after tilt training is increased by 30 or more than the pulse before the tilt training without reducing the blood pressure, when the pulse after tilt training is increased to 120 or higher, and when the pulse is reduced to 40 or less per minute after the tilt training.

When the risk of fainting of the patient is predicted, the process goes to step S335 to output a warning message notifying the fainting possibility of the patient through the output unit. For example, an alarm sound is ringed so that the other surrounding family members may perceive. Further, a warning message may be output to a predetermined terminal.

If the risk of fainting of the patient is not predicted, the process goes to step S340 to identify whether the patient maintains the posture at the time of starting the training. For example, when a changed amount of the measurement value of the sensor after starting the training exceeds a predetermined threshold value, it is determined that the posture at the time of starting the training is changed.

When the posture at the time of starting the training is changed, the process goes to step S345 to output a warning message.

When the posture at the time of starting the training is changed, it may determine whether the patient has fainted. For example, when the changed amount of the measurement value of the acceleration sensor is a predetermined limit value, it may be determined that a rapid fall has occurred in the patient. In another modified example, when the measurement values of the blood pressure, the pulse, the respiration, and the heart rate reach a predetermined limit value together with the changed amount of the measurement value, it is determined that the patient has fainted.

When it is determined that the posture at the time of starting the training is maintained in step S340, the process goes to step S350 to identify whether the set training time ends.

When the set training time has not ended, the process returns to step S330, and when the set training time has ended, the process goes to step S360 to store a training history. The training history may include a training date, a training start time, a training progress time, and a tilt angle.

For now, the present disclosure has been described with reference to the exemplary embodiments. It is understood to those skilled in the art that the present disclosure may be implemented as a modified form without departing from an essential characteristic of the present disclosure. Therefore, the disclosed exemplary embodiments may be considered by way of illustration rather than limitation. The scope of the present disclosure is presented not in the above description but in the claims and it may be interpreted that all differences within an equivalent range thereto may be included in the present disclosure.

1. A self-tilt training apparatus, comprising:
a storage unit which stores a daily training setting value including a training tilt angle and a training time of a patient;
a first sensor unit which includes a plurality of sensors in close contact with the patient and measures the tilt angle of the patient and a motion of the patient;
a control unit which determines whether the patient stands up according to a set training tilt angle at the time of starting the training and whether the patient maintains the tilt angle during the training time on the basis of the daily training setting value and the measurement values of the first sensor unit; and
an output unit which outputs a message, through any one or more of sight, touch, and hearing, according to a direction of the control unit.

2. The self-tilt training apparatus according to claim 1, further comprising:
a communication unit which transmits a message to a predetermined terminal according to a direction of the control unit,
wherein the control unit compares the matching of a tilt angle measured when the training starts and a set training tilt angle, and outputs a message including a comparison result to any one or more of the communication unit and the output unit.

3. The self-tilt training apparatus according to claim 2, wherein the control unit determines a fainting state of the patient during the training time on the basis of the measurement value of the first sensor unit, and outputs a message including a determination result through any one or more of the communication unit and the output unit.

4. The self-tilt training apparatus according to claim 3, further comprising:
a second sensor unit which measures any one or more of a blood pressure, a pulse, an electrocardiogram, a brainwave, and a cerebral blood flow of the patient,
wherein the control unit predicts a risk of fainting of the patient on the basis of the measurement value of the second sensor unit, and outputs a message including a prediction result to any one or more of the communication unit and the output unit.

5. The self-tilt training apparatus according to claim 4, wherein when it is determined to correspond to any one or more of cases when the blood pressure corresponds to a predetermined abnormal state, when a pulse corresponds to a predetermined abnormal state, when the electrocardiogram is not a sinus rhythm, when a brain wave changes from an alpha wave to a theta wave or a delta wave, and when a mean velocity of the cerebral blood flow is reduced to 30% or more, the control unit predicts that the patient has a risk of fainting.

6. The self-tilt training apparatus according to claim 5, wherein a case when the blood pressure corresponds to a predetermined abnormal state is any one of cases when a systolic blood pressure after the tilt training is reduced by 20 or more than the systolic blood pressure before the tilt training, when a systolic blood pressure after the tilt training is lower than 90, and when a diastolic blood pressure after the tilt training is reduced by 10 or more than a diastolic blood pressure before the tilt training, and
a case when the pulse corresponds to a predetermined abnormal state includes a case when the pulse within 10 minutes after tilt training is increased by 30 or more from the pulse before the tilt training without reducing the blood pressure, a case when the pulse after tilt training is increased to 120 or more, and a case when the pulse is reduced to 40 or less per minute after the tilt training.

7. The self-tilt training apparatus according to claim 2, further comprising:
an input unit which receives information from the patient,
wherein the storage unit acquires and stores a daily training setting value from any one of the communication unit and the input unit.

8. The self-tilt training apparatus according to claim 2, wherein the control unit records a training time and a training tilt angle generated according to the tilt training of the patient at every date in the type of calendar to output the training time and the training tilt angle through any one or more of the communication unit and the output unit.

9. The self-tilt training apparatus according to claim 2, wherein when a training time included in the daily training setting value is not fulfilled, the control unit outputs a message for encouraging the training through any one or more of the communication unit and the output unit.

10. The self-tilt training apparatus according to claim 4, wherein the first sensor unit includes a gyro sensor and an acceleration sensor, and the second sensor unit includes a vibration sensor, an acoustic sensor, a pulse measurement sensor, an electrocardiogram sensor, a blood pressure sensor, a respiration measurement sensor, an electroencephalogram sensor, and a cerebral blood flow sensor.

11. The self-tilt training apparatus according to claim 1, wherein the control unit outputs any one or more of a predetermined music and a motivation comment which are stored in advance during the training time through the output unit.

12. A self-tilt training method by an apparatus carried by a patient, comprising:
acquiring and storing a daily training setting value including a training tilt angle and a training time of the patient;
measuring a tilt angle of the patient and a motion of the patient by a first sensor unit including a plurality of sensors in close contact with the patient; and
determining whether the patient stands up according to a set training tilt angle at the time of starting the training and whether the patient maintains the tilt angle during the training time on the basis of the daily training setting value and the measurement values of the first sensor unit.

13. The self-tilt training method according to claim 12, wherein in the determining, the matching of a tilt angle measured when the training starts and a set training tilt angle is compared, and a message including a comparison result is output through any one or more of the communication unit and the output unit.

14. The self-tilt training method according to claim 13, wherein in the determining, a fainting state of the patient during the training time is determined on the basis of the measurement value of the first sensor unit, and a message including a determination result is output to any one or more of the communication unit and the output unit.

15. The self-tilt training method according to claim 14, wherein the measuring of a motion of the patient further includes:
measuring any one or more of a blood pressure, a pulse, an electrocardiogram, a brainwave, and a cerebral blood flow by a second sensor unit including one or more sensors, and
in the determining, a risk of fainting of the patient is predicted on the basis of the measurement value of the second sensor unit, and a message including a prediction result is output to any one or more of the communication unit and the output unit.

16. The self-tilt training method according to claim 15, wherein in the determining, when it is determined to correspond to any one of cases when the blood pressure corresponds to a predetermined abnormal state, when a pulse corresponds to a predetermined abnormal state, when the electrocardiogram is not a sinus rhythm, when a brain wave changes from an alpha wave to a theta wave or a delta wave, and when a mean velocity of the cerebral blood flow is reduced to 30% or more, it is predicted that the patient has a risk of fainting.

17. The self-tilt training method according to claim 12, further comprising:
when a training time included in the daily training setting value is not fulfilled, outputting a message for encouraging the training through any one or more of the communication unit and the output unit.
